(19) United States
(12) Patent Application Publication
(10) Pub. No.: US 2020/0230357 A1
(43) Pub. Date: Jul. 23, 2020

(54) EXPANDABLE INTRODUCER

(71) Applicant: TC1 LLC, Pleasanton, CA (US)

(72) Inventors: Keif Fitzgerald, San Jose, CA (US); Mike Butler, Dublin, CA (US); Paul Muller, San Carlos, CA (US)

(21) Appl. No.: 16/739,446

(22) Filed: Jan. 10, 2020

Related U.S. Application Data
(60) Provisional application No. 62/793,589, filed on Jan. 17, 2019.

(51) Int. Cl. A61M 25/00; A61L 29/04; A61L 29/08; A61L 29/14

(52) U.S. Cl. CPC A61M 25/0074; A61M 25/0045; A61M 25/0054; A61M 25/007; A61M 1/1008; A61L 29/085; A61L 29/14; A61M 2205/0266; A61L 29/042

(57) ABSTRACT

Disclosed herein is an expandable introducer for a catheter. The expandable introducer generally includes an elongate, tubular body extending between a proximal end and a distal end along a longitudinal axis. The elongate, tubular body generally includes an inner layer, and an outer layer where the inner layer and the outer layer form a wall structure configured to radially expand and contract as a medical device passes along the axis. At least one of the inner layer and the outer layer includes an elastomer. In some embodiments, the expandable introducer includes a reinforcing strut sandwiched between the inner and outer layers to provide additional mechanical benefits.

### CROSS REFERENCE TO RELATED APPLICATIONS

This application claims priority to provisional application Ser. No. 62/793,589, filed Jan. 17, 2019, which is incorporated by reference herein in its entirety.

### FIELD OF THE DISCLOSURE

The present disclosure relates generally to medical devices that are used in the human body. In particular, the present disclosure relates to an expandable introducer for use with a catheter or other medical devices to improve the overall performance of the catheter or other medical devices during a medical procedure.

### BACKGROUND

Heart disease is a major health problem that claims many lives per year. After a heart attack or other major cardiac event, a small number of patients can be treated with medicines or other non-invasive treatment. A significant number of other patients can recover from a heart attack or other cardiac event if provided with mechanical circulatory support in timely manner.

In one conventional approach for treating patients, a blood pump is inserted into a heart chamber, such as into the left ventricle of the heart and the aortic arch, to assist the pumping function of the heart. Other known conventional applications involve providing for pumping venous blood from the right ventricle to the pulmonary artery for support of the right side of the heart. The object of the pump is to reduce the load on the heart muscle for a period of time allowing the affected heart muscle to recover and heal. Blood pumps may also be used in some cases for percutaneous coronary intervention. In some cases, surgical insertion may potentially cause additional stresses in heart failure patients.

When a catheter is inserted into the body of a patient, an introducer, typically formed of a thin walled polymeric tube having a constant diameter, is placed through the site of the incision directly into the blood vessel. The catheter is then inserted (i.e., introduced) through the introducer into the blood vessel. After the catheter has been extended to its target location, the introducer often remains in place until the catheter is removed. In some instances, this can be several hours or even several days. Because the introducer is sized at least as large as the catheter, it may potentially in some cases partially occlude the blood vessel in which it remains. Additionally, some of the components of the catheter or the medical device attached thereto are of a size that they could potentially in some cases occlude the blood vessels in which it passes. This creates a difficulty in that the introducer must be large enough to allow passage of the components of the catheter while being small enough so that it does not occlude the blood vessel in which it remains. As such, there is a need for an introducer that is large enough to allow passage of the largest components of the catheter while being small enough to not occlude the blood vessel while remaining in place.

### BRIEF SUMMARY OF THE DISCLOSURE

In one embodiment, the present disclosure is directed to an expandable introducer for a catheter. The expandable introducer comprises: (i) an elongate, tubular body extending between a proximal end and a distal end along a longitudinal axis, the elongate, tubular body comprising: (a) an inner layer; and (b) an outer layer. The inner layer and the outer layer form a wall structure that is configured to radially expand and contract as a medical device passes along the longitudinal axis, and at least one of the inner layer and the outer layer comprises an elastomer.

In another embodiment, the present disclosure is directed to an expandable introducer for a catheter. The expandable introducer comprises: (i) an elongate, tubular body extending between a proximal end and a distal end along a longitudinal axis, the elongate, tubular body comprising: (a) an inner layer; (b) an outer layer; and (c) a nitinol reinforcing strut member located between the inner layer and the outer layer. The inner layer and the outer layer form a wall structure that is configured to radially expand and contract as a medical device passes along the longitudinal axis, and the inner layer and the outer layer each comprise an elastomer.

In another embodiment, the present disclosure is directed to an expandable introducer for a catheter. The expandable introducer comprises: (i) an elongate, tubular body extending between a proximal end and a distal end along a longitudinal axis, the elongate, tubular body comprising: (a) an inner layer; (b) an outer layer; and (c) a nitinol reinforcing strut member located between the inner layer and the outer layer. The inner layer and the outer layer form a wall structure that is configured to radially expand and contract as a medical device passes along the longitudinal axis, and the inner layer and the outer layer are fused together and are each formed from a urethane.

### BRIEF DESCRIPTION OF THE DRAWINGS

FIG. 1 illustrates a partial cut-away view of one embodiment of an expandable introducer that includes a reinforcing strut.

FIG. 2A illustrates a partial cut-away view of an embodiment of an expandable introducer that includes oval or ellipsoid shaped openings in the inner layer of the wall structure of the expandable introducer and does not include a reinforcing strut; FIG. 2B illustrates a cross-section of the expandable introducer of FIG. 2A; and FIGS. 2C, 2D, 2E, and 2F illustrate partial cut-away views of further embodiments of an expandable introducer that does not include a reinforcing strut but includes different shaped openings in the inner layer of the wall structure of the expandable introducer: 2C: diamond shaped, 2D: dog bone shaped, 2E: channel shaped, and 2F: spiral channel shaped.

FIG. 3 is a cross sectional view of the tubular body wall of one embodiment of an expandable introducer that includes a reinforcing strut.

FIG. 4 illustrates a catheter configuration that includes a percutaneous heart pump and is suitable for use with the expandable introducer described herein.

FIG. 5 illustrates a typical percutaneous heart pump in its expanded configuration.

FIG. 6 is a graph illustrating the austenite-martensite transition of a typical shape memory alloy.

### DETAILED DESCRIPTION OF THE DISCLOSURE

When a catheter is introduced into to the body of a patient during a medical procedure, an introducer or introducer sheath may also be used to aid the medical professional in placing and directing the catheter to the desired location in the vasculature. The introducer should be appropriately sized and configured so that any medical device that is part of the catheter can pass therethrough without substantial difficulty or damage. While the catheter is in place in the patient, the introducer remains in the blood vessel where it was initially placed in order to aid in removal of the catheter and medical device. However, as noted the diameter of the introducer should be such that the introducer does not significantly occlude the blood vessel in which it resides.

Embodiments of the present disclosure include an expandable introducer that radially expands to accommodate the passage of a medical device having a larger diameter than the unexpanded expandable introducer; that is, embodiments disclosed herein provide an expandable introducer that is capable of self-adjusting its diameter (i.e., localized expanding and contracting) to allow percutaneous medical devices having various diameters to be introduced into a blood vessel, such as an artery. After the medical device has passed through a point in the expanded configuration of the expandable introducer body, the expandable introducer body radially contracts back to a smaller configuration. The smaller configuration (or non-expanded configuration) is smaller and potentially significantly smaller, than the blood vessel in which it remains until final removal. The smaller configuration of the expandable introducer allows the passage of blood while it remains in the blood vessel, and thus minimizes blood occlusion and potential damage. The expandable introducers of the present disclosure may be particularly useful when the medical device being delivered into the vasculature of a patient is a percutaneous heart pump having a relatively large diameter, although other medical devices may be safely used as well in combination with the expandable introducer of the present disclosure and are suitable in accordance with the numerous embodiments described herein.

The expandable introducers of the present disclosure include introducers wherein the entire length of the introducer is capable of providing localized expansion and contraction as described herein. In these embodiments, the entire length of the introducer is configured to allow for the self-adjustment of its diameter. The expandable introducers of the present disclosure also include introducers where only a part or portion of the entire length of the introducer is capable of providing localized expansion and contraction as described herein. In these embodiments, for example, only a proximal portion or section (i.e., that portion or section that may reside in the femoral or other artery) of the introducer may provide expansion and contraction while a distal portion or section that typically resides in larger diameter arteries could remain at a constant diameter (no localized expansion and contraction). In other embodiments, multiple discreet parts or portions of the introducer may be capable of localized expansion and contraction.

In one embodiment of the present disclosure, disclosed herein is an expandable introducer for a catheter. The expandable introducer generally includes an elongate, tubular body extending between a proximal end and a distal end along a longitudinal axis. The elongate tubular body is constructed so as to include an inner layer and an outer layer (which, in some embodiments as noted hereinbelow, may be a single, fused layer at one or more locations along its length), where the inner layer and the outer layer form a generally cohesive and substantially fluid impermeable wall structure that is configured to radially expand and contract (localized expansion/contraction) as a medical device passes along its longitudinal axis. As note, in some embodiments, the inner layer and the outer layer may be fused together at one or more discrete locations such that the inner and the outer layers include both fused areas and areas that are not fused. In other embodiments, the inner layer and the outer layer may be substantially completely fused together such that there are no areas of the inner layer and the outer layers that are not fused together. In still other embodiments, the outer layer may be an un-slit tubular member that provides fluid impermeability and compressive force and that is fused in discrete, discontinuous locations to the inner layer. In many embodiments, the inner layer provides sufficient axial strength so that the component may be introduced into a patient and is configured such that it is a low friction, expandable inner layer to allow for easy insertion and passage of a medical device. In many desirable embodiments, the inner layer and the outer layer form a fluid impermeable structure.

At least one of the inner layer and the outer layer includes an elastic and/or pseudoelastic material. The inner layer and/or the outer layer may be completely formed of an elastic and/or pseudoelastic material, or may be partially formed from an elastic or pseudoelastic material. In some embodiments, both the inner layer and the outer layer include an elastic and/or pseudoelastic material. The inner layer and/or the outer layer may be formed from a single layer of material, or may be formed from multiple layers of materials. In some embodiments, the wall structure of the expandable introducer further includes a reinforcing strut disposed or sandwiched between all, or one or more portions, of the inner and outer layers. The reinforcing strut may provide numerous important benefits to the expandable introducer as discussed in more detail herein. In many embodiments of the present disclosure, the expandable introducer may be sized and configured to expand to allow a medical device having at its largest point a diameter of 8 Fr, or even 10 Fr, or even 12 Fr, or even 14 Fr or more to pass therethrough, as discussed hereinbelow.

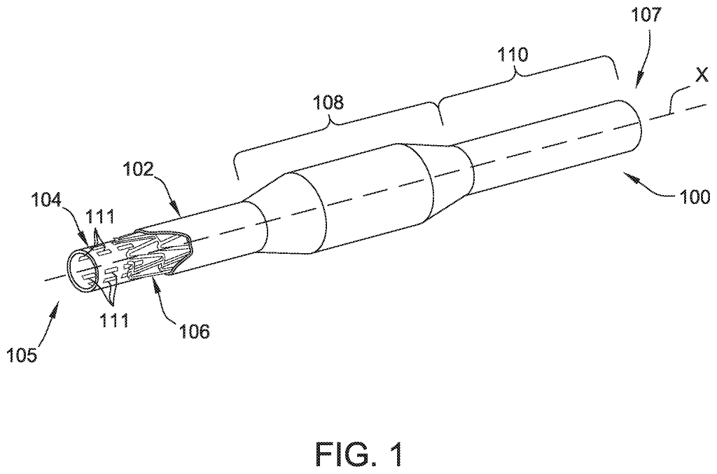

In reference to FIG. 1, shown therein is a partial cut-away view of one embodiment of an expandable introducer 100 for use with a catheter (not shown in FIG. 1) in accordance with the present disclosure. Expandable introducer 100 includes an outer layer 102, an inner layer 104, and a reinforcing strut 106 disposed between outer layer 102 and inner layer 104. Expandable introducer 100 also has a proximal end 105 and a distal end 107. Inner layer 104 includes openings 111 cut therein and further discussed hereinbelow. Although illustrated in FIG. 1 and generally discussed herein as including a reinforcing strut (106 in FIG. 1), it will be recognized by one of skill in the art based on the disclosure herein that expandable introducers of the present disclosure may be suitably prepared without such a reinforcing strut. Further, it is within the scope of the present disclosure for such a reinforcing strut to be present on only a portion, or only on multiple portions, of the wall structure formed by the inner and the outer layers. In some embodiments, reinforcing strut 106 is in the shape of a rolled up or tubular mesh or braid material (such as a nitinol or another memory shape alloy material), although other configurations of reinforcing strut 106 may be suitable as well in many embodiments.

Referring again to FIG. 1, outer layer 102 and inner layer 104 expand and contract with reinforcing strut 106 as a medical device (not shown in FIG. 1) passes along longitudinal axis X of expandable introducer 100. The expanded configuration 108 of expandable introducer 100 is sufficiently large to accommodate a variety of medical devices (not shown in FIG. 1) that may be used during a medical procedure as noted above. After the medical device moves along the longitudinal axis of expandable introducer 100, the region of localized expansion 108 reverts back to its contracted configuration 110. Contracted configuration 110 may also be commonly referred to as the insertion or smaller configuration as it is the configuration of the expandable introducer when it is first inserted into a blood vessel of the patient. As such, expandable introducer 100 is able to safely allow passage therethrough of medical devices having a diameter larger than the diameter of its contracted configuration. As will be recognized by one skilled in the art based on the disclosure herein, outer layer 102, inner layer 104 and reinforcing strut 106 may be sized, configured, and constructed so as to control the force required to expand expandable introducer 100 as described herein. The materials for the construction of outer layer 102, inner layer 104 and/or reinforcing strut 106 may be selected based on one or more properties thereof so as to control the forces required to expand expandable introducer 100, depending upon the intended use or application of the medical device. Additionally, the thickness of the materials selected for the construction of outer layer 102, inner layer 104, and reinforcing strut 106 may be chosen so as to control the force desired for expansion to occur.

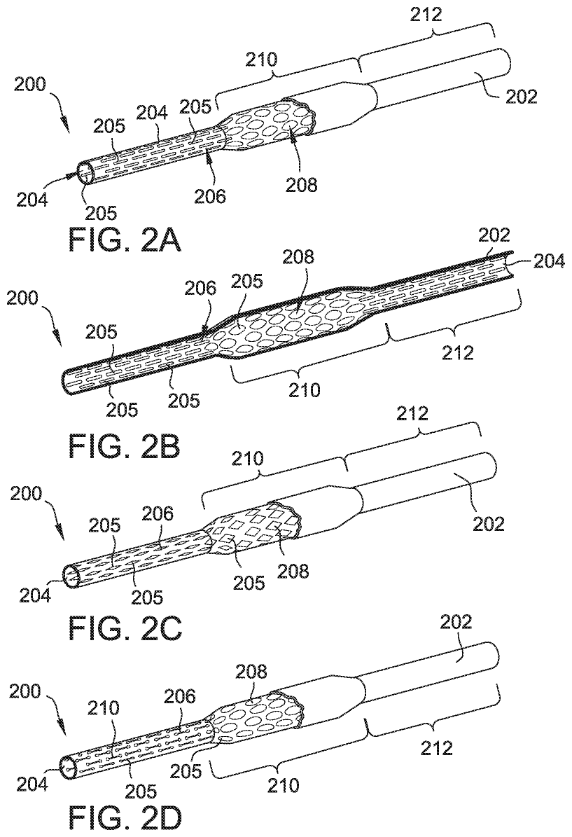

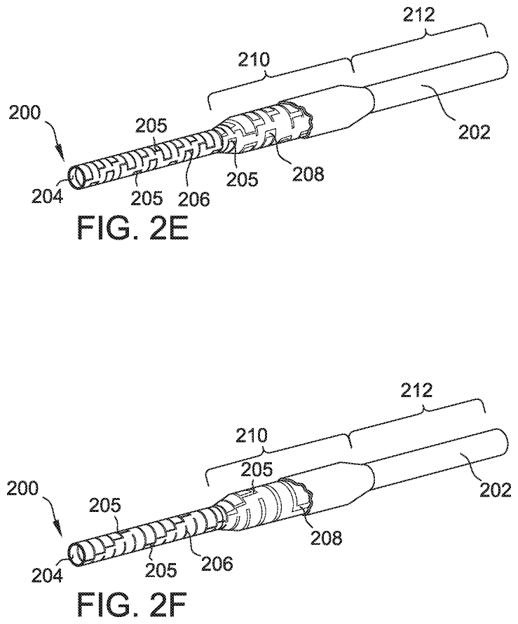

In reference to FIGS. 2A to 2F, shown therein are other embodiments of an expandable introducer 200 for use with a catheter (not shown in FIGS. 2A to 2F) in accordance with the present disclosure. Expandable introducer 200 includes an outer layer 202, an inner layer 204, and a plurality of openings 205 in inner layer 204 to enhance the stretchability of inner layer 204. In some embodiments (not shown in FIGS. 2A-2F), openings 205 may extend through both outer layer 202 and inner layer 204. In other embodiments (as illustrated in FIGS. 2A-2F), openings 205 only extend through inner layer 204 (and not outer layer 202) to increase the fluid impermeability of the structure. Openings 205 can be in a closed configuration 206 (i.e., in an unexpanded or relaxed state without any forces acting thereon) or an open configuration 208 (i.e., in an expanded state with forces acting thereon to produce the expanded state). As a medical device (not shown in FIGS. 2A-2F) passes axially along expandable introducer 200, the body (i.e., the tubular wall formed of outer layer 202 and inner layer 204) of expandable introducer 200 expands into an expanded configuration 210 which is accommodated by the expansion of openings 208. When the medical device moves axially through the expandable introducer, the region of localized expansion 210 reverts back to its contracted configuration 212 and the openings in the body of expandable introducer 200 revert to their closed configuration 206. Different shapes of the openings are possible, including, but not limited to those shown in FIGS. 2A-2F. FIGS. 2A (illustration of a partial cut away view) and 2B (illustration of a cross sectional view of 2A) show a slit shape that expands into an oval or ellipsoid shape for the open configuration of the openings in the outer layer while FIG. 2C shows a diamond shape; FIG. 2D shows a dog bone shape; FIG. 2E shows a channel shape, and FIG. 2F shows a spiral channel shape. Other shapes for the openings are possible. The examples of different shapes for the openings in the inner surface (and/or the outer surface and/or a reinforcing strut, as discussed below) of the tubular wall structure shown here are non-limiting and for illustration purposes only. In some embodiments, the opening may be in the form of a diamond, an oval, a channel or series of channels, a circle, a rectangle, a square, a triangle, a star, a pentagon, a hexagon, a polygon, an irregular polygon, an ellipse, a trapezoid, a rhombus and combinations thereof. The openings on the inner layer and the outer layer (and/or the reinforcing strut as described below) may or may not have the same shape, and may or may not be present along the entire length of the inner layer and the outer layer (and/or the reinforcing strut).

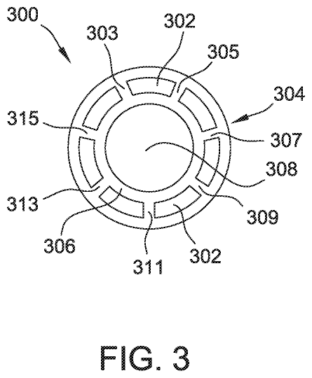

Now referring to FIG. 3, there is illustrated a cross-sectional view of an expandable introducer 300 in accordance with one alternative embodiment of the present disclosure. Expandable introducer 300 includes inner layer 306, outer layer 304, reinforcing strut 302, and lumen 308. In the illustrated embodiment, inner layer 306 and outer layer 304 may be fused or joined together at various fuse points 303, 305, 307, 309, 311, 313, and 315 to sandwich reinforcing strut 302 therebetween in a liquid-tight structure. As noted and as further discussed hereinbelow, reinforcing strut 302 may be sized and configured to deliver numerous advantages to the expandable introducer.

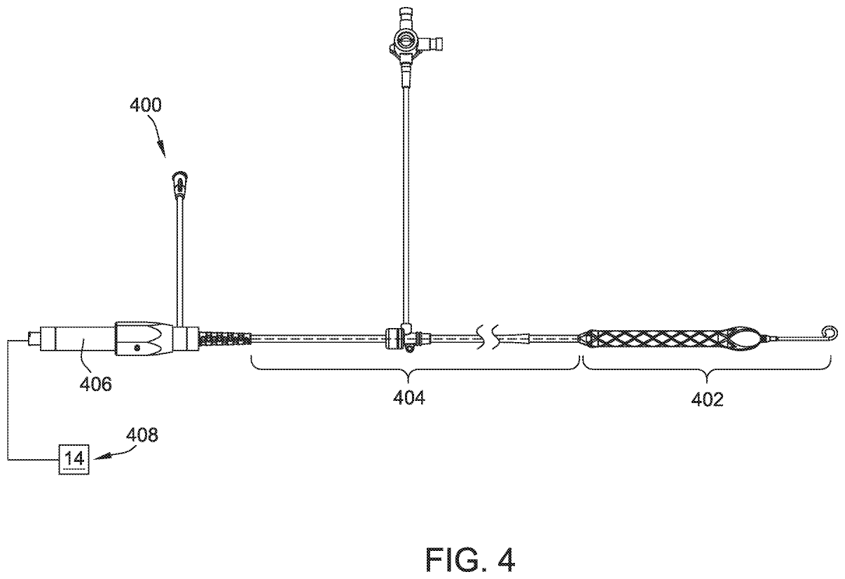

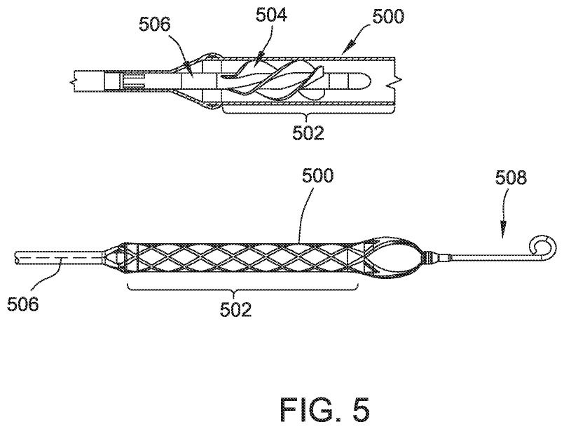

The expandable introducers of the present disclosure are suitable for use in combination with any number of catheters and catheter systems. One nonlimiting example of a catheter system 400 including a medical device 402 that is suitable for use with an expandable introducer is shown in FIG. 4. In general, catheter system 400 includes a catheter shaft 404 having a proximal and distal end where the medical device 402 is positioned on the distal end. Catheter system 400 includes a handle 406 with which to control and manipulate catheter shaft 404 and medical device 402. The handle may optionally be connected to an additional controller 408. A more detailed view of medical device 402 is illustrated in FIG. 5.

With reference to FIG. 5, illustrated therein is a percutaneous heart pump 500 that may be introduced into a blood vessel of a patient by use of an expandable introducer as described herein. Heart pump 500 includes an elongate body that surrounds and encompasses an impeller 504 driven by an impeller shaft 506 that runs from, and is controlled by, catheter handle 406 (See FIG. 4). The distal end of heart pump 500 includes an atraumatic tip 508 in order to facilitate insertion of the pump after passing through the expandable introducer. Although body 502 of the heart pump 500 collapses into a smaller configuration during insertion, in some embodiments, heart pump 500 does not collapse down to a diameter smaller than the blood vessel in which it must traverse or be inserted. Due to the material used to manufacture impeller 504, it also may not collapse down as small as the smallest blood vessels in which it must traverse or be inserted. As such, all embodiments of the expandable introducer disclosed herein temporarily radially expand to accommodate the passage of heart pump 500 and then revert to their original and smaller diameter after the pump traverses axially through lumen 308 of the expandable introducer.

As discussed above, in many embodiments described herein, the expandable introducers may desirably include a reinforcing strut located between the inner layer and the outer layer to improve overall performance of the expandable introducer. The reinforcing strut (or reinforcing member) may provide a number of desirable mechanical properties to the expandable introducer including, for example, radial force, axial stiffness, kink resistance, and the like. While providing these desirable properties, the reinforcing strut still allows localized expansion (and contraction) of the expandable introducer as a larger section of a medical device passes therethrough as discussed herein.

Although numerous commercially available materials may be particularly suitable for construction of the reinforcing strut, in many embodiments, shape memory alloys (SMA) are suitable for use in construction of the reinforcing strut. The SMA may suitably be in a tubular mesh or tubular braid in many embodiments. SMAs are metallic alloy materials that have the ability to “memorize” or retain its previous shape when subjected to certain stimuli, such as stress or heat. An SMA material, like nitinol, may also possess superelasticity that allows a component including such a material to exhibit pseudo-elastic recovery or “memory” from one shape to another multiple times upon the application and release of deforming stress or force. A small stress or force may induce considerable deformation, but the material or component including such a material recovers its original shape when the deforming force or stress is released. There is no need for any other stimulus, such as heating or cooling, for the deformed material to return to its original shape. The superelasticity of such an SMA material is a mechanical type of shape memory that is utilized for making an expandable introducer exhibiting reversible expanding and collapsing configurations. Under applied force or stress, such as when a medical device is moved axially through the expandable introducer, the expandable introducer wall structure radially expands to its expanded configuration. Because it is fabricated from an SMA, it “memorizes” its original shape and returns to its original shape once the medical device advances axially through the lumen. In one specific embodiment, the reinforcing strut may be formed from nitinol formed into a tubular shape having a small diameter with strut features that would bend and allow expansion into a larger diameter.

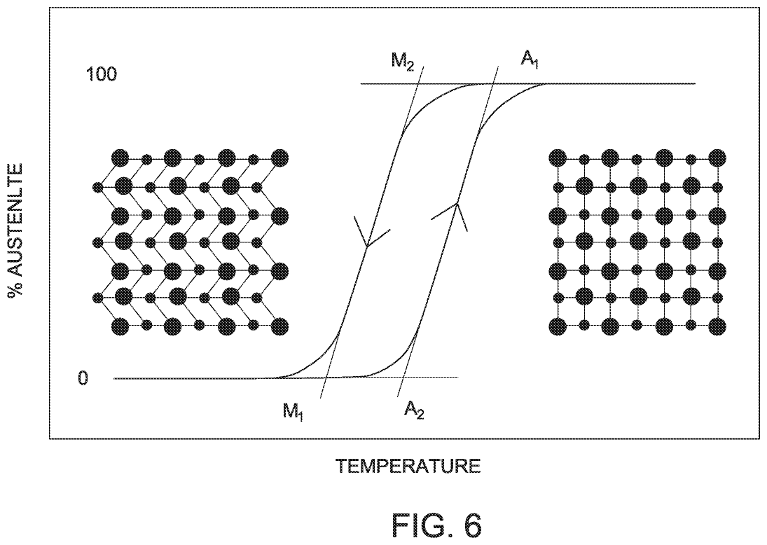

SMAs generally display two distinct crystal forms: martensite, primarily with variant sheared platelets, and austenite (the parent or memory phase), with long-range order. The martensite form of an SMA material is self-accommodating and deforms by a so-called twining mechanism that transforms different sheared platelet variants into a variant accommodating to the maximum deformation in the direction of the applied force. At low temperatures, an SMA material may exist as martensite that can be deformed by a relatively small force. In contrast, at high temperatures, the material may exist as austenite which is hard to deform like normal metals. Therefore, upon thermal stimulus (heating or cooling), an SMA material may undergo a phase transformation as the temperature increases or decreases. For example, when heated, an SMA material that exists as martensite (e.g., at ambient or body temperature) may start to undergo the phase transformation-to-austenite at a so-called “Austenite-Start temperature” (Aₛ or A₂) and finish the transformation at a relatively high, so-called “Austenite-Finish temperature” (A_{f} or A₁), above which the material exists as austenite (i.e., the parent or memory phase), displaying shape memory. Similarly, upon cooling, an SMA material that exists as austenite may start to undertake the transformation-to-martensite at a so-called “Martensite-Start temperature” (Mₛ or M₂) and finish the transformation at a relatively lower so-called “Martensite-Finish temperature” (M_{f} or M₁), below which the material exists as martensite, exhibiting shape recovery. Such phase transformations induced by thermal stimulus is illustrated in the graph in FIG. 6. Due to material hysteresis, phase transformation temperatures at which an SMA material may exist at a similar or comparable phase are not equal. For example, Mₛ and A_{f} (or M_{f} and Aₛ) at which a SMA material exists in similar phases are not equal. In general, an “Austenite-Finish temperature” (A_{f}) is utilized to characterize the shape memory and hyperelasticity effects of an SMA material.

In addition to thermal stimulus, phase transformation-to-martensite or phase transformation-to-austenite of an SMA material may take place under other stimulus, such as applied force or stress. For example, for an SMA material that exists as austenite at the temperature of interest that is slightly below its active “Austenite-Finish temperature” A_{f} (or comparably “Martensite-Start temperature” Mₛ), applied stress may “force” the material to undergo the phase transformation-to-martensite, at which the material would exhibit considerable deformation for a relatively small applied force or stress. Once the force or stress is released, the material in martensite reverts back to austenite and recovers its original shape (the memory phase). Such phase transformation-to-martensite effect as induced by external force or stress makes an SMA material appears to be extremely elastic, and is known as superelasticity. This superelasticity is used for the selection of SMA materials for fabricating the expandable introducers described herein.

Examples of SMA materials include, but are not limited to, nickel-titanium (nitinol), copper-zinc, copper-zinc-aluminum, copper-aluminum-nickel, and gold-cadmium. In some embodiments, the SMA used to make the reinforcing strut is nickel-titanium. For a typical SMA material, its active A_{f} varies based on the exact composition of the material. In some embodiments, the expandable introducer includes an SMA having an active austenite finish temperature (A_{f}) that is near or below the body temperature of the patient. In humans, that temperature is generally about 98.6° F. or 37° C. In some embodiments, the A_{f} of the SMA is from 0° C. and 35° C. In yet another embodiment, the A_{f} is from 5° C. to 30° C. In yet another embodiment, the A_{f} is from 10° C. to 25° C. In yet another embodiment, the A_{f} is from 10° C. to 20° C. In some embodiments, the reinforcing strut of the expandable introducer includes nitinol.

In accordance with the present disclosure, at least one of the inner layer and the outer layer that form the tubular wall structure of the expandable introducer includes an elastic or pseudoelastic (elastomer) material. Both the inner layer and the outer layer may be a single layer or may be formed from multiple layers that may or may not be fused together to form a single, integral wall structure. In some embodiments, the inner layer will include an elastomer, while in other embodiments the outer layer will include and elastomer. In still other embodiments, both the inner and the outer layer will include an elastomer. The inner layer and/or the outer layer may be formed exclusively from an elastomer, or may be partially formed from an elastomer in combination with one or more other materials. In those embodiments where the inner wall and/or the outer wall is formed in whole or in part from a material other than an elastomer, suitable conventional materials such as thin walled polymeric tubes and the like may be used. Because the outer layer of the expandable introducer will be in physical contact with at least one part of the body of the patient, the elastomer is desirably biocompatible for at least the duration during which it is in use or inside the body of the patient. In some embodiments, the inner layer also includes an elastomer that is optionally biocompatible. For the elastomer of the inner layer and the outer layer, each elastomer is independently selected from the group consisting of Ecoflex®, Thoralon®, silicon elastomer, butadiene rubber, ethylene-propylene (EPM) elastomer, ethylene-propylene-diene terpolymer (EPDM) elastomer, styrene butadiene styrene block polymers, ethylene propylene copolymers, PTFE, polyimides, ethylene propylene diene copolymers, ethylene vinyl alcohol polymer, and combinations thereof. In some embodiments, the elastomer is Ecoflex®. In other embodiments, the elastomer is Thoralon®. Any biocompatible elastomer may be used for the preparation of the inner and/or outer layers of the expandable introducer.

The thickness of the wall structure of the tubular body of the expandable introducer is at least a combination of the thickness of the outer layer, the inner layer, and, when present, the reinforcing strut. The thickness of the walls should be sufficient to provide suitable strength such that it does not rupture during use, especially during expansion and contraction. Other layers, in addition to the inner layer, the outer layer, and the reinforcing strut may also optionally be present to provide additional beneficial properties to the expandable introducer. Other layers, when present, may add strength, improve kink resistance and/or may improve the elasticity of the structure. Additionally, the thickness of the walls must be thin enough such that the expandable introducer is flexible so that it can be inserted into a patient's blood vessel without causing undue irritation or trauma to any vascular surfaces in which it comes into contact. In some embodiments, the thickness of the wall structure of the expandable introducer is from about 25 μm to about 2500 μm, from about 50 μm to about 1750 μm, from about 100 μm to about 2000 μm, from about 150 μm to about 1500 μm, or from about 200 μm to about 1250 μm. In some embodiments, the thickness of the wall structure of the expandable introducer is about 50 μm, about 100 μm, about 200 μm, about 250 μm, about 300 μm, about 400 μm, about 500 μm, about 600 μm, about 700 μm, about 800 μm, about 900 μm, about 1000 μm, about 1200 μm, about 1400 μm, about 1600 μm, about 1800 μm, about 2000 μm, or about 2500 μm. About in this context means ±10%. In some embodiments, the inner layer may have a thickness of from about 50 p.m to about 100 μm, the outer layer may have a thickness of from about 75 μm to about 125 p.m, and the reinforcing strut, when present, may have a thickness of from about 75 μm to about 125 μm.

As discussed hereinabove, the expandable introducer disclosed herein has at least two different configurations—the expanded configuration and the contracted configuration (alternatively referred to as the smaller or original configuration) (See, for example, FIG. 2A). During insertion of the expandable introducer into a blood vessel and prior to insertion of the medical device, the expandable introducer is in its contracted configuration. When the medical device is inserted into the expandable introducer, if the device has a larger diameter than the expandable introducer, the expandable introducer radially expands into an expanded configuration. There is an upper limit to the amount of expansion possible for the expandable introducer. The diameter of the medical device is desirably at or below this upper limit. The upper limit to the expansion can be determined either by measuring the absolute size (e.g., a measured diameter) or by comparison to the contracted configuration (e.g., a percent increase). The size of a medical device is often measured in units called French or French Gauge.

The table below illustrates a comparison between French and other units. In some embodiments, the expandable introducer as described herein will expand to its upper limit as compared to its contracted configuration by about 10%. By way of illustration and not limitation, if the contracted configuration of the expandable introducer had a diameter of 10 French, then an expansion of 10% means that the upper limit to expansion is 11 French. If the contracted configuration of the expandable introducer has a diameter of 20 mm, then an expansion of 10% means that the upper limit of expansion is 22 mm. When the amount of expansion of the expandable introducer is referenced as a percentage, the units are generally either French, millimeters or inches.

In some embodiments of the present disclosure, the expandable introducer will expand to its upper limit as compared to its contracted configuration by about 20%, by about 30%, by about 40%, by about 50%, by about 60%, by about 70%, by about 80%, by about 90%, by about 100%, by about 150%, by about 200%, by about 250%, by about 300%, by about 350%, by about 400%, or by about 500%. In other embodiments, the expandable introducer will expand from a collapsed configuration of 5 French to an expanded configuration of 20 French. In some embodiments, the expandable introducer will expand from a collapsed configuration of 6 French to an expanded configuration of 18 French. In other embodiments, the expandable introducer will expand from a collapsed configuration of 7 French to an expanded configuration of 16 French. In some other embodiments, the expandable introducer will expand from a collapsed configuration of 8 French to an expanded configuration of 14 French. It is understood that simply because the expandable introducer can expand up to a certain upper limit, during use, the medical device may be of a size such that full expansion is not necessary when used. These values describe the capabilities of the expandable introducer, not a limit or requirement when in use.

| French | Diameter | Diameter |
|---|---|---|
| Gauge | (mm) | (inches) |
| 3 | 1 | 0.039 |
| 4 | 1.333 | 0.053 |
| 5 | 1.667 | 0.066 |
| 6 | 2 | 0.079 |
| 7 | 2.333 | 0.092 |
| 8 | 2.667 | 0.105 |
| 9 | 3 | 0.118 |
| 10 | 3.333 | 0.131 |
| 11 | 3.667 | 0.144 |
| 12 | 4 | 0.158 |
| 13 | 4.333 | 0.170 |
| 14 | 4.667 | 0.184 |
| 15 | 5 | 0.197 |
| 16 | 5.333 | 0.210 |
| 17 | 5.667 | 0.223 |
| 18 | 6 | 0.236 |
| 19 | 6.333 | 0.249 |
| 20 | 6.667 | 0.263 |
| 22 | 7.333 | 0.288 |
| 24 | 8 | 0.315 |
| 26 | 8.667 | 0.341 |
| 28 | 9.333 | 0.367 |
| 30 | 10 | 0.393 |
| 32 | 10.667 | 0.419 |
| 34 | 11.333 | 0.445 |

The expandable introducers of the present disclosure may be manufactured or fabricated by any suitable method that allows for the production of a multi-layer medical device including the materials and having the characteristics as described herein. In one exemplary manufacturing embodiment, an expandable introducer is fabricated and includes an inner layer and an outer layer wherein the inner layer is initially in a small, closed diameter configuration and the outer layer is applied over the inner layer to form the multi-layer device. In this embodiment, any desired pattern (dog bone, diamond etc.) is first cut into the inner layer, which may generally be formed from a tube or a sheet that is rolled into a tube. Once the pattern profile is cut into the inner layer, the elastic outer layer is applied over the inner layer (in some embodiments, the outer layer may also have a desired pattern cut into it at a desired time during the manufacturing process). The outer layer may be applied over the inner layer in any suitable manner including, for example, by inserting the inner layer into an outer layer tube, by spraying an outer layer over the inner layer (such that when the spray dries, it forms the outer layer), and/or by dipping the inner layer into a solution that, when dried, forms the outer layer. In some embodiments, once the inner layer and the outer layer are formed, a hemostatic valve or similar valve may be attached to the proximal end of the structure to provide additional benefits.

In another embodiment as described herein, the expandable introducer may be fabricated so as to include a reinforcing strut located or sandwiched between the inner layer and the outer layer. In this embodiment, the expandable introducer may be fabricated by first cutting any desired pattern (dog bone, diamond etc.) into the reinforcing strut (which may be, for example, a wire-braided tube or sheet that is rolled into a tube and heat set) and the inner layer (in some embodiments, the outer layer may also have a desired pattern cut into it). The inner layer may then be inserted into the reinforcing strut and adhered to the strut material in one or more locations (such as by a heat treatment) such that expansion is not substantially impeded. The elastic outer layer is then applied over the inner layer as described above and further adhesion may be optionally formed. In some embodiments, once the inner layer and the outer layer are formed, a hemostatic valve or similar valve may be attached to the proximal end of the structure to provide additional benefits.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. An expandable introducer for a catheter, the expandable introducer comprising:
an elongate, tubular body extending between a proximal end and a distal end along a longitudinal axis, the elongate body comprising:
an inner layer; and
an outer layer;
wherein the inner layer and the outer layer form a wall structure that is configured to radially expand and contract as a medical device passes along the longitudinal axis; and
wherein at least one of the inner layer and the outer layer comprises an elastomer.

2. The expandable introducer of claim 1, wherein the wall structure further comprises a reinforcing strut member positioned between the inner layer and the outer layer.

3. The expandable introducer of claim 2, wherein the reinforcing strut comprises a shape memory alloy.

4. The expandable introducer of claim 3, wherein the shape memory alloy is nitinol.

5. The expandable introducer of claim 2, wherein both the inner layer and the outer layer comprise an elastomer.

6. The expandable introducer of claim 5, wherein both the inner layer and the outer layer comprise the same elastomer.

7. The expandable introducer of claim 2, wherein the elastomer is selected from the group consisting of a silicon elastomer, butadiene rubber, ethylene-propylene elastomer, ethylene-propylene-diene terpolymer elastomer, styrene butadiene styrene block polymer, urethane, ethylene propylene copolymer, ethylene propylene diene copolymer, ethylene vinyl alcohol polymer, and combinations thereof.

8. The expandable introducer of claim 7, wherein the elastomer is formed from a urethane.

9. The expandable introducer of claim 5, wherein the inner layer and the outer layer are formed from a urethane.

10. The expandable introducer of claim 2, wherein the elongate, tubular body of the introducer expands and contracts between about 5 and 25 French.

11. The expandable introducer of claim 2, wherein the elongate, tubular body of the introducer is sized and configured to expand to at least about 400%.

12. The expandable introducer of claim 2, wherein a thickness of the wall structure is from about 25 μm to about 2500 μm.

13. The expandable introducer of claim 12, wherein the thickness of the wall structure is from about 100 μm to about 1000 μm.

14. The expandable introducer of claim 1, wherein at least the inner layer of the elongate, tubular body comprises a plurality of openings configured to permit radial expansion and contraction of the introducer as a medical device passes along the longitudinal axis.

15. The expandable introducer of claim 14, wherein the plurality of openings are substantially the same size.

16. The expandable introducer of claim 14, wherein the shape of the plurality of openings are selected from the group consisting of a slit, a diamond, a dog bone, an oval, a circle, a rectangle, a square, a triangle, a star, a pentagon, a channel, a hexagon, a polygon, an irregular polygon, an ellipse, a trapezoid, a rhombus and combinations thereof.

17. An expandable introducer for a catheter, the expandable introducer comprising:
an elongate, tubular body extending between a proximal end and a distal end along a longitudinal axis, the elongate body comprising:
an inner layer;
an outer layer; and
a nitinol reinforcing strut member located between the inner layer and the outer layer;
wherein the inner layer and the outer layer form a wall structure that is configured to radially expand and contract as a medical device passes along the longitudinal axis; and
wherein the inner layer and the outer layer each comprise an elastomer.

18. The expandable introducer of claim 17, wherein the inner layer and the outer layer are comprised of a urethane.

19. The expandable introducer of claim 8, wherein at least the inner layer of the elongate, tubular body comprises a plurality of openings configured to permit radial expansion and contraction of the introducer as a medical device passes along the longitudinal axis.

20. An expandable introducer for a catheter, the expandable introducer comprising:
an elongate, tubular body extending between a proximal end and a distal end along a longitudinal axis, the elongate body comprising:
an inner layer including a plurality of openings;
an outer layer; and
a nitinol reinforcing strut member located between the inner layer and the outer layer;
wherein the inner layer and the outer layer form a wall structure that is configured to radially expand and contract as a medical device passes along the longitudinal axis; and
wherein the inner layer and the outer layer are fused together and are each formed from a urethane.
